(19) United States
(12) Patent Application Publication
(10) Pub. No.: US 2025/0241540 A1
(43) Pub. Date: Jul. 31, 2025

(54) ERGONOMIC HEART RATE TRACKING AND BIAS DETECTOR SYSTEM AND METHOD

(71) Applicant: Chet M. Pitts, Detroit, MI (US)

(72) Inventor: Chet M. Pitts, Detroit, MI (US)

(21) Appl. No.: 18/425,530

(22) Filed: Jan. 29, 2024

(51) Int. Cl. A61B 5/0205; A61B 5/00; A61B 5/024; A61B 5/0255

(52) U.S. Cl. CPC A61B 5/0205; A61B 5/0002; A61B 5/0255; A61B 5/4803; A61B 5/681; A61B 5/742; A61B 5/02438; A61B 2560/0223

(57) ABSTRACT

An ergonomic heart rate tracking and bias detecting system and method is provided. The system is configured to determine if users exhibit biases by measuring their heartrates and/or recording audio via the smartwatch during simulated tests or real-world situations. The system is intended to help those supervisors of employees by requiring employees to wear the smartwatch and using the system track and record their behavior. The system is configured to transmit data from the smartwatch to the system application for analysis to measure the heartrate of users. A low or resting heartrate may denote a pass situation, where a high heartrate may denote a failure situation, meaning a potential bias is detected in the user. Audio recorded by the system is also analyzed for tone and language to further determine the presence of bias in the user. Supervisors may then decide whether to hire/fire/or retrain the user.

### BACKGROUND

At present, the evaluation and hiring of employees is fraught with challenges. Due to decades of institutional procedures and systematic hiring processes, employees are often hired based strictly on procedures that do not include bias screening but rather focus on traditional testing which has failed to include, let alone focus, on biases both perceived and latent, within all applicants.

Even if employees are asked about their beliefs and feelings involving race and discrimination, privilege, and other biases, they are prepared for these questions in the application processes and therefore provide answers that they know are acceptable to the hiring agency.

Further, these questions involving bias often are in the form of written or oral examinations, not real-world situations involving members of the public. These sanitized simulations are, by definition, not variable and do not induce an instinctive reaction from the applicant that a dynamic interaction with the public would induce.

What is needed then, is a system and method to add a realistic and dynamic element to the hiring of employees and particularly employees who engage the public-in other words, this system and method could be applied to all employees at least at some level to determine any innate biases present in those applicants-and provide the hiring agency with data to train, exclude, or otherwise evaluate each applicant.

The system and method of the present disclosure includes a smart watch device (but may be incorporated in other devices) that is configured to record the heart rate and audio (speech) of the applicant/device wearer. The applicant/device wearer may wear the device during real and/or simulated tests and/or interactions with the public to gather data, which the system application (software) analyzes and provides the hiring agency with a report detailing any biases exhibited by the applicant.

### SUMMARY OF THE DISCLOSURE

The present disclosure over the problems and disadvantages associated with the current strategies and designs and provides new tools and methods for an ergonomic heart rate tracking system and method.

One embodiment of the present disclosure is directed to an ergonomic heart rate tracking and bias detector system and method (EHTS) of using the same that includes a heart rate tracking smart watch device with a heart rate tracking smart watch housing configured to engage the wrist of a user, where the heart rate tracking smart watch housing includes a heart rate tracker, an audio recorder, a display, a wireless communicator, and a computing device including a processor and memory communicatively connected to the heart rate tracker, the audio recorder, the display, and the wireless communicator, where the computing device is configured to receive an input activating the device using the display; record the heart rate data and the voice audio of the user in response to activation of the device; transmit the recorded voice audio and the recorded heart rate data of the user to an external device application, where in response to the transmission, the external device application is configured to generate a report on the user, ascertain whether the user is suitable for a particular job based on predetermined bias criteria; and receive an input deactivating the device using the display in response to the complete transmission of the recorded audio and the heart rate data to the application.

Another embodiment of the disclosure is directed to an ergonomic heart rate tracking and bias detector system and method that includes a heart rate tracking smart watch device where the computing device is further configured to execute a heart rate data calibration application when the device is activated using the display.

Another embodiment of the disclosure is directed to an ergonomic heart rate tracking and bias detector system and method that includes a heart rate tracking smart watch device where the computing device is further configured to display user information upon completion of the heart rate data calibration using the display.

Another embodiment of the disclosure is directed to an ergonomic heart rate tracking and bias detector system and method that includes a heart rate tracking smart watch device of where the computing device is further configured to record the heart rate data of the user during a bias simulation and transmit the heart rate data to the application.

Another embodiment of the disclosure is directed to an ergonomic heart rate tracking and bias detector system and method that includes a heart rate heart rate tracking smart watch device where the computing device is further configured to analyze the user's heart rate data according to predetermined parameters including levels of heart rate activity and transmit the analyzed data to the application.

Another embodiment of the disclosure is directed to an ergonomic heart rate tracking and bias detector system and method that includes a heart rate heart rate tracking smart watch device where the computing device is further configured to analyze the user's heart rate data according to predetermined parameters including levels of heart rate activity and provide the analyzed data to the user using the display.

Another embodiment of the disclosure is directed to an ergonomic heart rate tracking and bias detector system and method that includes a heart rate tracking smart watch device where the computing device is further configured to provide the heart rate data to the user using the display.

Another embodiment of the disclosure is directed to an ergonomic heart rate tracking and bias detector system and method that includes a heart rate tracking smart watch device where the computing device is further configured to indicate to the user that audio is being recorded using the display.

Another embodiment of the disclosure is directed to an ergonomic heart rate tracking and bias detector system and method that includes a heart rate tracking smart watch device where the computing device is further configured to transmit the recorded audio and the recorded heart rate data of the user to a virtual reality simulation application.

Another embodiment of the disclosure is directed to an ergonomic heart rate tracking and bias detector system and method that includes a heart rate tracking smart watch device where the computing device is further configured to provide to the user confirmation of the complete transmission of the recorded audio and heart rate data to the application using the display.

Another embodiment of the disclosure is directed to an ergonomic heart rate tracking and bias detector system and method that includes a heart rate tracking smart watch device where the application further is configured to generate a folder for a new user to store the recorded audio and the recorded heart rate data of the user to the ergonomic heart rate tracking system.

Another embodiment of the disclosure is directed to an ergonomic heart rate tracking and bias detector system and method that includes a heart rate tracking smart watch device where the application further comprises external memory storage to record data of each user, where the data includes heart rate data and audio files of the voice of each user.

Another embodiment of the disclosure is directed to an ergonomic heart rate tracking and bias detector system and method that includes a heart rate tracking smart watch device where the external device application is configured to analyze the voice audio files of the user for variations in pitch.

Another embodiment of the disclosure is directed to an ergonomic heart rate tracking and bias detector system and method that includes a heart rate tracking smart watch device where the heart rate tracker includes a user heart rate calibration mode.

Another embodiment of the disclosure is directed to an ergonomic heart rate tracking and bias detector system and method that includes a heart rate tracking smart watch device where the computing device is further configured to execute situational simulations on the user.

Another embodiment of the disclosure is directed to an ergonomic heart rate tracking and bias detector system and method that includes a heart rate tracking smart watch device where the computing device is further configured to stealth mode, where the stealth mode includes a limited display output to the user.

Another embodiment of the disclosure is directed to an ergonomic heart rate tracking and bias detector system and method that includes a heart rate tracking smart watch device where the computing device is further configured with a location device to transmit the location of the heart rate smart watch device to the external device application.

Another embodiment of the disclosure is directed to an ergonomic heart rate tracking and bias detector system and method that includes a heart rate tracking smart watch device where the computing device is further configured to retrieve the status of the user from the external device application and selectively record the heart rate and voice audio of the user dependent upon that status.

Another embodiment of the disclosure is directed to an ergonomic heart rate tracking and bias detector system and method that includes a method including receiving, using a heart rate tracking smart watch device, an input from a user activating the device using the display, wherein the input includes at least one of a character or a user biometric authenticator, recording, using the heart rate tracking smart watch device, the heart rate data and the audio of the user in response to activation of the device, transmitting, using the heart rate tracking smart watch device, the recorded audio and the recorded heart rate data of the user to an application, wherein the application stores the data and generates a report on the user and ascertains whether the user is suitable for a particular job based on predetermined bias criteria, and receiving, using the heart rate tracking smart watch device, an input from the user deactivating the device using the display in response to the complete transmission of the recorded audio and the heart rate data to the application.

Another embodiment of the disclosure is directed to an ergonomic heart rate tracking and bias detector system and method that includes a non-transitory computer-readable storage medium comprising instructions that cause one or more computing devices to receive, using a heart rate tracking smart watch device, an input from a user activating the device using the display, wherein the input includes at least one of a character or a user biometric authenticator, record, using the heart rate tracking smart watch device, the heart rate data and the audio of the user in response to activation of the device, transmit, using the heart rate tracking smart watch device, the recorded audio and the recorded heart rate data of the user to an application, wherein the application stores the data generates a report on the user and ascertains whether the user is suitable for a particular job based on predetermined bias criteria, and receive, using the heart rate tracking smart watch device, an input from the user deactivating the device using the display in response to the complete transmission of the recorded audio and the heart rate data to the application.

Other embodiments and advantages of the disclosure are set forth in the part in the description and accompanying drawings, which follow and are included herein, respectively, and in part, may be obvious from this description and accompanying drawings, or may be learned from practice of the disclosure.

### BRIEF DESCRIPTION OF DRAWINGS

The disclosure is described in greater detail by way of the example only and with referenced to the attached drawings, in which:

FIG. 1 is a perspective view of a smart watch and display screens of the smart watch, according to an embodiment of the present disclosure;

FIG. 2 is an array of display screens of a smart watch according to an embodiment of the present disclosure;

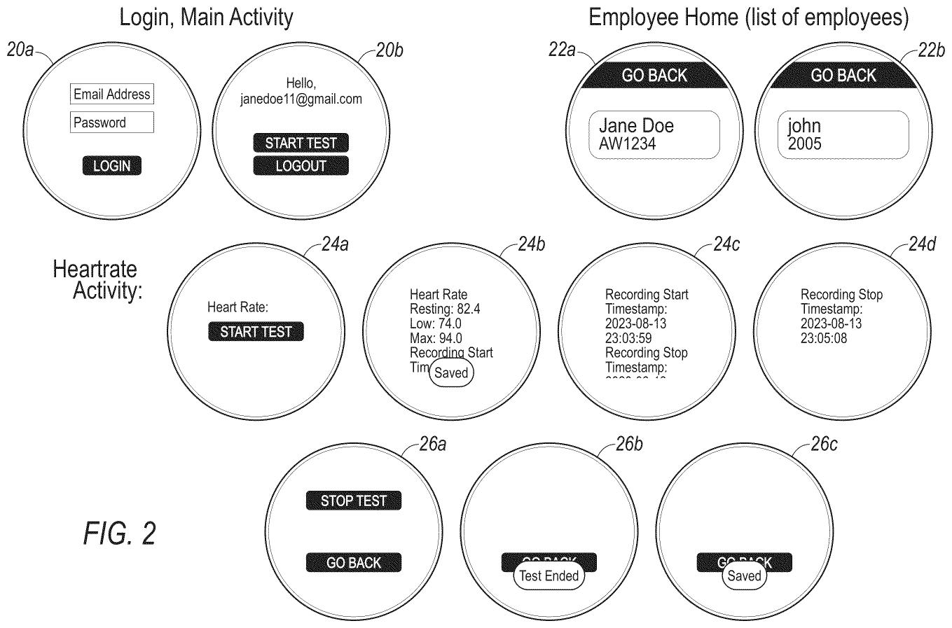

FIG. 3 is a flowchart according to an embodiment of the present disclosure;

FIG. 4 is a flowchart according to an embodiment of the present disclosure;

FIG. 5 is a flowchart according to an embodiment of the present disclosure;

FIG. 6 is a flowchart according to an embodiment of the present disclosure;

FIG. 7 is a flowchart according to an embodiment of the present disclosure;

FIG. 8 is a behavior model flowchart according to an embodiment of the present disclosure;

FIG. 9 is a flowchart of a mobile device and a smart watch according to an embodiment of the present disclosure;

FIG. 10 is a flowchart of the EHTS application according to an embodiment of the present disclosure;

FIG. 11 is an array of displays of the EHTS according to an embodiment of the present disclosure;

FIG. 12 is an array of displays of the EHTS according to an embodiment of the present disclosure;

FIG. 13 is an array of displays of the EHTS according to an embodiment of the present disclosure;

FIG. 14 is a pair of displays of the EHTS according to an embodiment of the present disclosure; and

FIG. 15 is an array of displays of the EHTS according to an embodiment of the present disclosure.

### DETAILED DESCRIPTION

As embodied and broadly described herein the Drawings, the present disclosure is directed to an ergonomic heart rate tracking system and method. In some embodiments, the system and method may be realized in a smart watch device with at least one application and may include an internet/cloud-based application to collect, analyze, and store data collected by the smart watch device.

The ergonomic heart rate tracking and bias detector system (EHTS) in some embodiments, includes at least one smart watch device (smart watch) having a processor, memory, and applications stored therein to perform various functions. The smart watch may also include a display, a power source, an audio recording device (e.g., a microphone), and heart rate tracking device (such as, but not limited to) a device that may scan the blood flow of the user near the user's wrist. The smart watch may also include wireless connectivity, such as Bluetooth® and/or internet (Wi-Fi), or other wireless connectivity to be developed.

The display may be a color display and may also be touch sensitive and/or be controlled by input devices (e.g., buttons) located on the smart watch. In other embodiments, other input/output devices are contemplated, such as, but not limited to, audio and remote commands, such as by another device or application.

The power source may be a battery, such as a lithium-ion battery. The battery may be charged wirelessly or using a cable connection to a power supply. In other embodiments, the smart watch may be at least partially charged by movement of the user. In other embodiments, the smart watch may also include solar power charging of the smart watch battery.

The heart rate tracking device may utilize light emitting diodes (LEDs) positioned on the smart watch to illuminate the user's wrist, called photoplethysmography. One or more optical sensors configured to receive the light from the LEDs transmit data based on the light to the smart watch device, wherein an application processes the data to determine the user's heart rate. In other embodiments, an electrocardiogram (ECG) device included in the smart watch device that may be configured to engage the wrist of the user. Other technologies are contemplated to measure the heart rate of the user that may be incorporated into the smart watch.

One purpose of the EHTS, including the smart watch device, is to provide a reliable and efficient system that can be used as a preventative measure to help select applicants for positions of authority and to measure existing employees in positions of authority for any biases they may consciously or unconsciously exhibit.

The EHTS may be used in the evaluation and hiring of new employees for positions of authority (or for other positions involving the public, or for employers generally). It is contemplated that EHTS may be used for private and public sector applicant and employee evaluation and/or training.

In embodiments, the EHTS may be used for training involving simulations of public interaction. Using a job applicant as an example, the applicant would place the EHTS smart watch device on one of their wrists and complete the initialization and calibration process (to be described in more detail below). The applicant will then be placed in a simulated work situation involving the public or co-workers. The simulation may involve virtual reality (VR), using goggles or other equipment, or the simulation may involve actors in a live-action simulation (or role-play).

In embodiments, at least one application is included in the EHTS. The application (or applications) may be based on or be an Android or iPhone application (or another application for electronic devices to be developed). The application may include capability to transmit, receive and store data from users of the EHTS including data collected from the smart watch of the EHTS.

The application may be configured to allow for user profile and account settings to be used across multiple users. For example, an employer, such as a retailer or food service establishment, may have a number of EHTS including smart watches in their inventory and assign them to one or various users at different times (such as during work shifts).

The application may be configured to generate one or more reports based at least in part on data collected by the EHTS including, but not limited to, smart watches of the EHTS. The reports may include other data inputted by the employer, employee, or applicant. The reports generated by the EHTS may be transmitted or stored via the internet/cloud services for use by the employer or other authorized users.

The smart watch may be configured to make audio recordings of the user. These recordings may be commenced automatically, by the application of the EHTS. For example, the EHTS may communicate with the smart watch of the user and based at least upon several parameters, operate the audio recording device of the smart watch.

In one embodiment, the user may login and place the smart watch on their wrist at the beginning of their work shift. Based at least in part on location data and whether or not the user is working or on break, the audio device of the smart watch may record their audio (heart rate and other attributes in some embodiments). If the user is dispatched on a work-related task, for example, the audio device of the smart watch may be activated by the EHTS to begin recording the user's audio (and those of others within range of the smart watch). Upon arrival of the user, the audio device captures the voice (including tone and stress attributes) of the user, as well as those at the location and transmits that data to the EHTS for analysis and evaluation.

The EHTS then processes the audio recordings of the user, analyzing the audio recordings based on predetermined qualifications and attributes in the EHTS software to develop a report on the incident to determine if the user exhibited any bias toward any of the individuals involved.

In another embodiment, the user may login and place the smart watch on their wrist at the beginning of their work shift. Based at least in part on location data and whether or not the user is on break or at work, the heart rate tracker of the smart watch may record their heart rate (and other attributes). If the user is dispatched to a work activity, for example, the heart rate device of the smart watch may be activated by the EHTS to begin recording the user's heart rate. Upon arrival of the user, the heart rate device captures the heart rate of the user and transmits that data to the EHTS for analysis and evaluation.

In another embodiment of the EHTS, web-integrated software of the EHTS stores employee/user data collected from at least the smart watch device. The EHTS software also includes user profile and account settings to be established and maintained for storing data, analyzing that data, and generating reports based at least upon parameters entered in the EHTS software. Reports can be generated in real-time, based on historical data, or a combination of both.

The EHTS software is configured to receive data from the smart watch, including heart rate of the user during a simulation (or test) or from real-life interactions with the public or other employees (e.g., casino workers). The EHTS software (or in some embodiments, the smart watch device) evaluates at least three levels of heart rate data. For example, the three levels of heart rate data may include Level 1—low heart rate/at rest (Pass); Level 2—normal heart rate (Pass); and Level 3—accelerated heart rate (Fail).

The EHTS software is configured to store, analyze, and process audio (e.g., voice) data from the user (and in some embodiments, also the smart watch). The audio data may be collected during simulations, testing, or from real-life situations. Additionally, the EHTS software is configured to receive information about the user, either from the user or from an employer, such as a human resource or hiring manager. Various settings are also included, such as activation time of the smart watch. Activation may be location-based, time-based, or activity-based, in some embodiments, or a combination thereof. For example, and without limitation, a user's smart watch may activate when their shift begins and deactivate when their shift ends. In another example, a factory or hospitality user worker's smart watch may activate when they are on the factory floor or interacting with resort guests.

The EHTS software is configured to connect with and communicated via the internet and/or other wireless communication to connect to the smart watch of the EHTS. The EHTS software and the smart watch may then synchronize and transfer data between the EHTS software and the smart watch as necessary or determined by account settings of the EHTS.

In another embodiment, the EHTS may be utilized by a human resource administrator as follows. The administrator has full access to the EHTS software and the smart watch(es). The administrator also has access to all data and analyses generated by the EHTS and the smart watches worn by authorized users. Each user, for example employees or trainees, may establish or be assigned an account in the EHTS using the smart watches or via administrator actions.

The EHTS may be used in a simulated work activity, such as an unsatisfied customer situation. The simulation will be used to evaluate current employees or trainees. In the case of current employees, the evaluation may involve retraining (if the employee scores poorly), termination if the employee scores very badly, or no action if the employee scores above a predetermined threshold that may be set by the administrator. In the case of the trainees, the evaluation may involve additional training (if the trainee scores poorly), termination if the cadet trainee scores very badly, or no action if the trainee scores above a predetermined threshold that may be set by the administrator.

In the simulation, either the employee or the trainee is assigned a smart watch of the EHTS. After logging into the EHTS and placing the smart watch on their wrist, calibration may occur. Once the initial set up is complete, the simulation may begin.

The employee or trainee is dispatched to a real or simulated work situation. The dispatcher provides them (either the employee or trainee) with information about the work situation, including location and the racial information of the participants. The EHTS via the smart watch, records their heart rate and audio after receiving the dispatch and detailed information. This first step in the simulation allows for valuable data to be recorded. The heart rate and audio can reveal any biases present in the employee or trainee even at the initial dispatch—based at least in part on the racial information and the location—as this information can affect the employee or the trainee. This information is transmitted to the EHTS application for analysis and report generation. Before the employee or trainee arrives at the scene of the work situation, the EHTS can make an initial determination on whether any biases exist. The EHTS, via communication with the smart watch and the application, receives heart rate data and/or audio data, and via the application software determines whether the employee or trainee passes the test.

In the case of a second real work situation, there may be some differences including the races of some or all the individuals involved, the location, or other variables. The dispatcher provides them (either the employee or trainee) with information about the work situation, including location, racial information, other variables. The EHTS via the smart watch, records their heart rate and audio after receiving the dispatch and detailed information. This first step in the simulation allows for valuable data to be recorded. The heart rate and audio can reveal any biases present in the employee or trainee even at the initial dispatch—based at least in part on the racial information and the location—as this information can affect the employee or the trainee. This information is transmitted to the EHTS application for analysis and report generation. Before the employee or trainee arrives at the scene of the work situation, the EHTS can make an initial determination on whether any biases exist. The EHTS, via communication with the smart watch and the application, receives heart rate data and/or audio data, and via the application software determines whether the employee or trainee passes the test.

In the case of another simulated work situation, such as a dissatisfied customer, there may be some differences including the races of some or all the individuals involved, the location, or other variables. The dispatcher provides them (either the employee or trainee) with information about the work situation, including location, racial information, other variables. The EHTS via the smart watch, records their heart rate and audio after receiving the dispatch and detailed information. This first step in the simulation allows for valuable data to be recorded. The heart rate and audio can reveal any biases present in the employee or trainee even at the initial dispatch—based at least in part on the racial information and the location—as this information can affect the employee or the trainee. This information is transmitted to the EHTS application for analysis and report generation. Before the employee or trainee arrives at the scene of the work situation, the EHTS can make an initial determination on whether any biases exist. The EHTS, via communication with the smart watch and the application, receives heart rate data and/or audio data, and via the application software determines whether the employee or trainee passes the test.

Additional real or simulated work situations (or a combination thereof) may be used to test employees or trainees (or other employees, associates, team members, etc.) to determine whether any biases exist.

In another embodiment of the disclosure, an active employee is equipped with a smart watch including the EHTS application (user facing) which is connected to the EHTS (human resources facing) via the Internet or other wireless connection.

The employee activates the EHTS via the interface on the smart watch. Activation may be in a number of forms, such as entering user ID and password, biometric login (fingerprint, voice, etc.), or a combination of login methods. Activation may occur automatically once the employee work shift begins-due to synchronization between the EHTS and the employer's roster.

During the employee's work shift (on-duty), the EHTS tracks the employee's heart rate and/or audio, particularly but not necessarily only, when the employee is working. The EHTS can be configured to monitor the employee throughout the entirety of the employee's shift, including time not working—such as during a work break, while driving a work vehicle, while on walking in a particular location, such as a neighborhood, special event, or as a security function (e.g., an airport). As a result, the EHTS may be used to determine the employee's level of bias while observing members of the public as well as those engaged while at work.

At the end of the employee's shift, the employee clocks off duty and the EHTS can be deactivated by the employee or by the EHTS administrator (human resources, etc.). Data from the EHTS, including packets of data containing the employee's heart rate and/or audio is transmitted to the EHTS application on the administrator side for analysis. In other embodiments, some or all of the data analysis of the employee's heart rate and audio may occur on the smart watch. Transmission of the analyzed data to the EHTS administrator account is used to generate a report(s) on the employee's performance on the shift. In other embodiments, the EHTS of the smart watch and EHTS of the administrator may communicate in real-time (or approximately real-time) based on available communication systems and methods to provide the administrator with immediate analysis of the employee's performance with particular focus on any biases exhibited by the employee.

In the case of real-time or near real-time bias analysis by the EHTS, the administrator (such as a shift supervisor) can use the EHTS to defuse a possible undesired situation. The EHTS, through the heart rate tracking and audio recording of the smart watch and communication with the administrator (via the EHTS admin interface), a determination of a bias-influenced situation can be quickly addressed. The administrator can communicate with the employee to inform and educate the employee on the bias determination by the EHTS as well as take other corrective action. The EHTS can be configured to recommend solutions based on the bias determination and analysis of the employee's behavior. The administrator, taking this data into account, can make decisions about how to address the situation. For example, one solution could be to dispatch one or more employees to the situation who have, via the EHTS, exhibited positive EHTS analysis results (i.e., showed little or no bias towards the public).

In embodiments, the EHTS includes a plurality of smart watches, each with its own password. EHTS administrators may have sole access to the smart watches, and via a secure interface may enter their administrator personal indication number (PIN). The administrator logs into their EHTS account on the smart watches and determines which employee is being tested with the EHTS. The administrator gives (assigns) one of the smart watches to the employee to wear on their wrist during their work shift.

The assigned smart watch, now on the employee's wrist, records their heart rate and/or audio during their work shift. The employee will not be aware of the smart watch's heart rate tracking and/or audio recording. In embodiments, the smart watch display will remain blank, or simply display the current time or other information not related to the EHTS running on the smart watch. At the end of the employee's shift, or when the administrator decides the employee's test is complete, the EHTS application is halted and the data is uploaded from the smart watch to the administrator's EHTS application, which may be accessed via mobile application on a smart device, computer, or a smart watch. The EHTS, accessed by the administrator, analyzes the heart rate and/or audio of the employee and displays the results to the administrator. Based on the report generated by the EHTS for the administrator, the administrator may then decide on the employee's biases based on the EHTS analysis. For example, no bias detected, low heart rate—no action required; medium heart rate—corrective action recommended; high heart rate—immediate correction required.

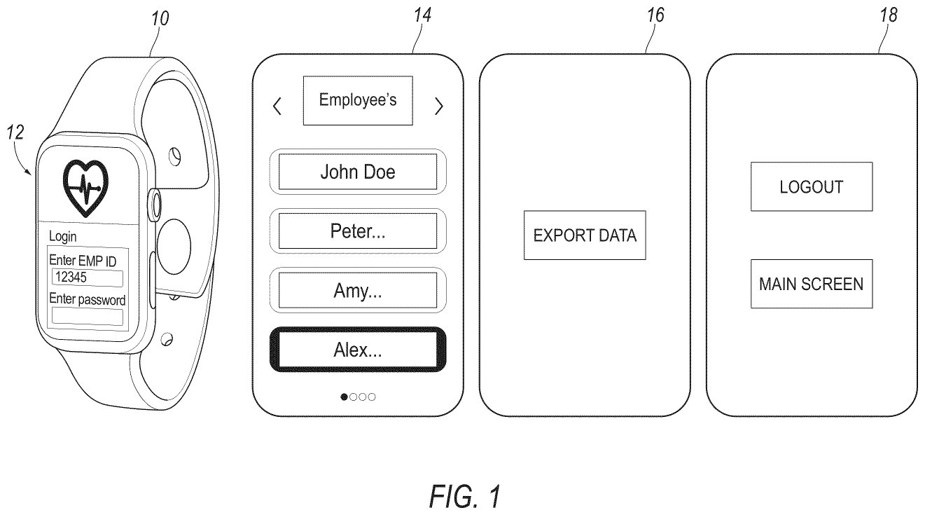

Referring now to FIG. 1, one embodiment of the disclosure is shown. A smart watch 10 is shown having a display 12. The smart watch may also include a heart rate tracking device and an audio recording device (as described herein).

The display 12 of the smart watch 10 may include a plurality of screens including a login screen 14 for administrator access. The display may be touch-sensitive (or accessible via a plurality of present techniques or to be developed). A listing of employees may be accessed by the EHTS administrator. The administrator may then scroll through the existing employee names to arrive at the employee who is to be assigned the smart watch 10, or the administrator may enter a new employee's name if it is not already present. Another screen the administrator may access is the Export Data screen 16. Using the screen, the administrator may export heart rate and/or audio data recorded by the smart watch 10 after being worn by an employee. Yet another screen shown by the display 12 of the smart watch 10 to the administrator is a logout/main screen 18.

In another embodiment of the display 12 of the smart watch 10 of the EHTS, a login screen 20a is shown. A user, either an employee/wearer of the smart watch 10 or the EHTS administrator, may enter their email address and password to access the smart watch 10. Once the user enters their email and password, a second screen 20b is shown, welcoming the user to the EHTS and providing buttons, including at least Start Test and Logout buttons. The display 12 may further provide to the user employee home screens 22a, 22b that may provide to the user employee information, including name and/or ID number. Further still, display 12 of the smart watch may provide a plurality of heart rate screens 24a-24d. Screens 24a-24d may show the user heart rate information, such as resting heart rate, lowest heart rate measured, highest (or max) heart rate measured, and other heart rate tracking info, such as recording information/duration of both heart rate and/or audio of the user. Yet another display 12 of the smart watch may provide a plurality of testing related screens 26a-26c. These screens 26a-26c may show the user functions of the smart watch 10 of the EHTS function buttons such as Stop Test (to stop a EHTS testing session), Go Back (to return to another screen), as well as display other information such as Test Ended (signifying a completed or to-be ended EHTS test) and Saved (signifying that heart rate and/or audio data from a test of the user by the EHTS has been saved (locally and/or to the EHTS application accessible by the administrator of the EHTS.

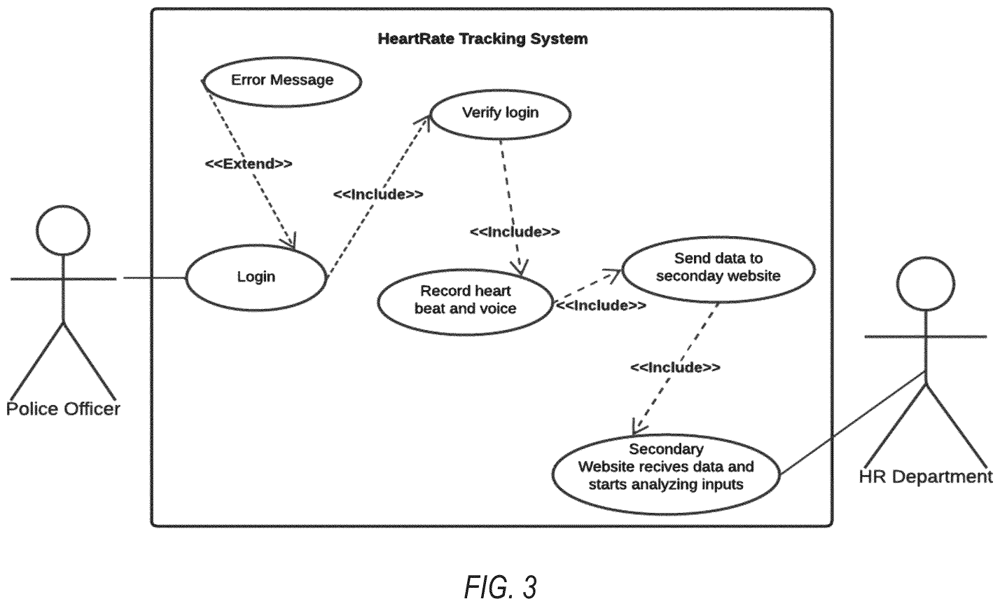

FIG. 3 illustrates one embodiment of the EHTS of the present disclosure. An employee, in this example, given a smart watch 10 that is running an application of the EHTS, enters their login information. The login information may include their username/email address, password or PIN, or use other methods to authenticate the identity of the user, such as biometric data. If the login information is entered incorrectly, an error message displayed. If the login information is entered correctly, the smart watch 10 may then be configured to record the heart rate and/or audio of the user. At a future time, such at the end of the user's work shift, the smart watch 10 may be configured to upload or transmit the recorded heart rate and/or audio data of the user to the EHTS website or application. The administrator of the EHTS, such as the human resources (HR) department may then access the user's EHTS data and reports generated by the EHTS for evaluation of the user by the administrator.

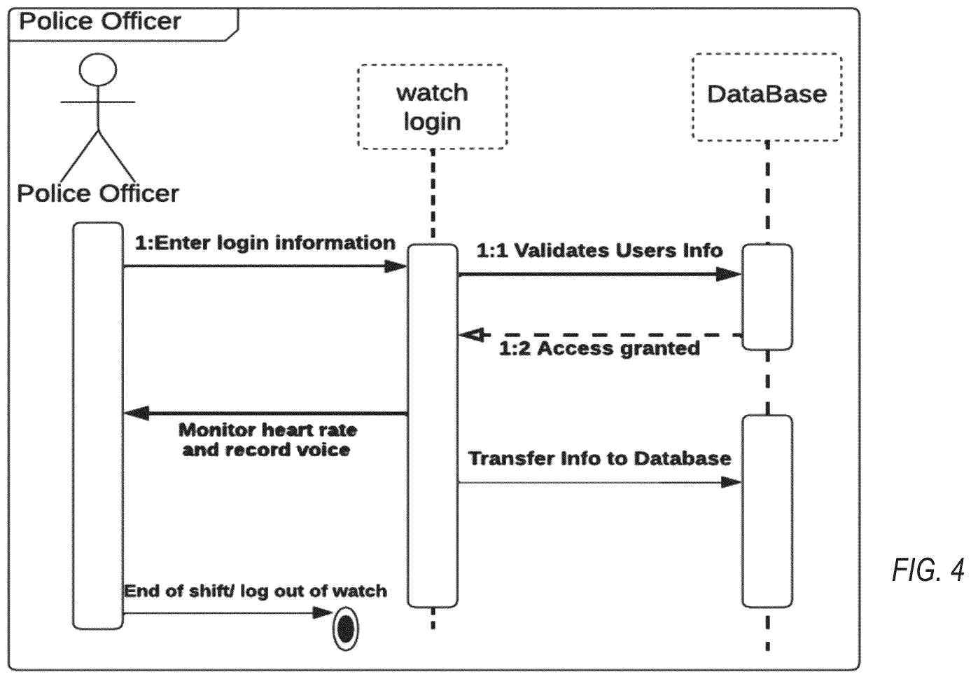

FIG. 4 illustrates another embodiment of the operation of the EHTS according to the present disclosure. An employee is assigned or provided a smart watch 10 that includes the EHTS and its related software and hardware as described herein. The employee enters their login information, such as at the beginning of their shift. A database of the EHTS, which may be located in the EHTS software running on a mobile device, a cloud application, a server, or a combination thereof, validates the employee's user information. If the user information is validated, access to the EHTS is granted. The smart watch 10 monitors the heart rate and records the audio of the employee during their shift and transfers (transmits) via Bluetooth®, the internet, or other wireless communication to the EHTS database for storage, analysis, and report generation. At the end of the employee's shift, the employee logs out of the smart watch 10.

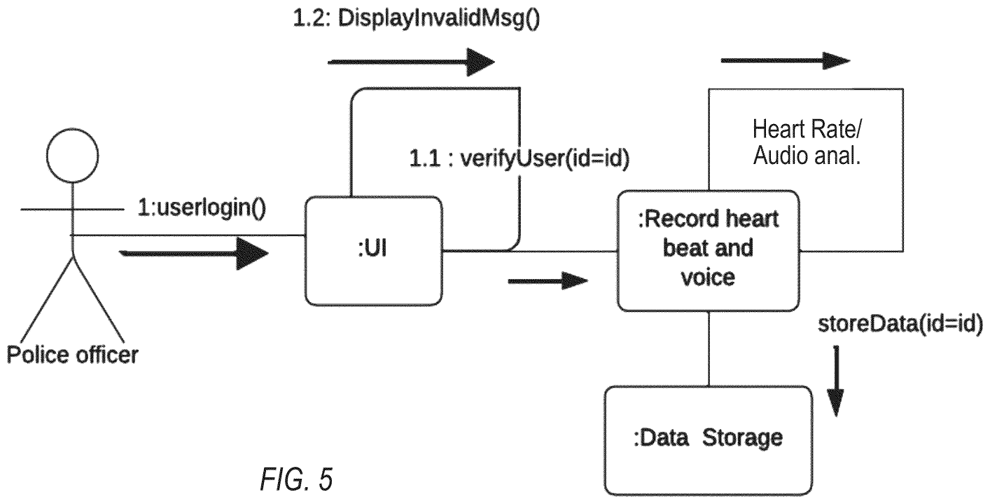

FIG. 5 illustrates an embodiment of the EHTS application. An employee user logs into the EHTS (using smart watch 10). The login information is entered via the User Interface (UI) and the user data packet is analyzed by the EHTS to verify whether the user has account in the EHTS or if incorrect data is entered (whereupon the EHTS displays an invalid user/account info via a data packet to the UI of the display 12 of the smart watch 10. The EHTS records the heart rate (beat) and the user's voice generating additional data packets that are transmitted via the smart watch to the data storage of the EHTS. Additionally, and/or optionally, the heart rate and/or audio of the user may be analyzed by the EHTS either automatically (to generate a report) or at the option of the administrator.

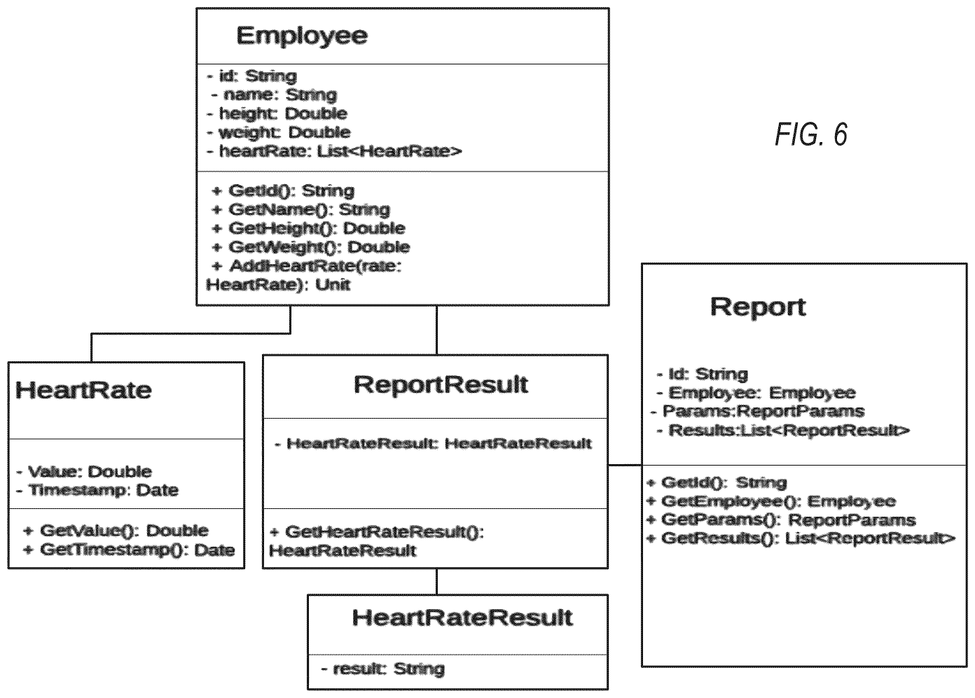

FIG. 6 details the operation of the EHTS software application, which may be included in smart watch 10 and/or a mobile application used on a computing device such as a mobile phone or computer. Heart rate data collected from the user, along with employee data is processed by the EHTS to generate a heart rate result for a test period (such as a work shift or another predetermined period of time). The EHTS outputs a heart rate analysis result; for example, resting heart rate; low heart rate=pass; high heart rate=fail. The EHTS may also generate a report that includes employee data combined with the heart rate data, for evaluation by the administrator using the EHTS.

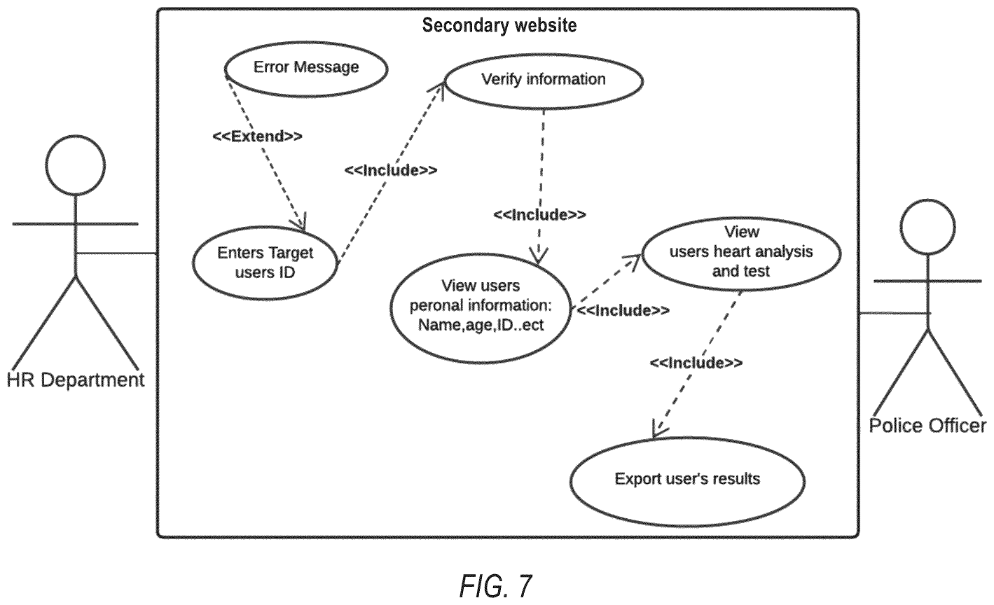

FIG. 7 illustrates another embodiment of the EHTS. A human resource (HR) administrator logs into EHTS via the website interface and enters an employee (target) identification. If improper or incorrect information is entered, EHTS outputs an error message. EHTS verifies correct employee information entered by the administrator, displaying the employee's personal information that also may include any heart rate and audio data collected by EHTS. The administrator may then use EHTS to export the employee's results for review and evaluation of any biases present in the employee.

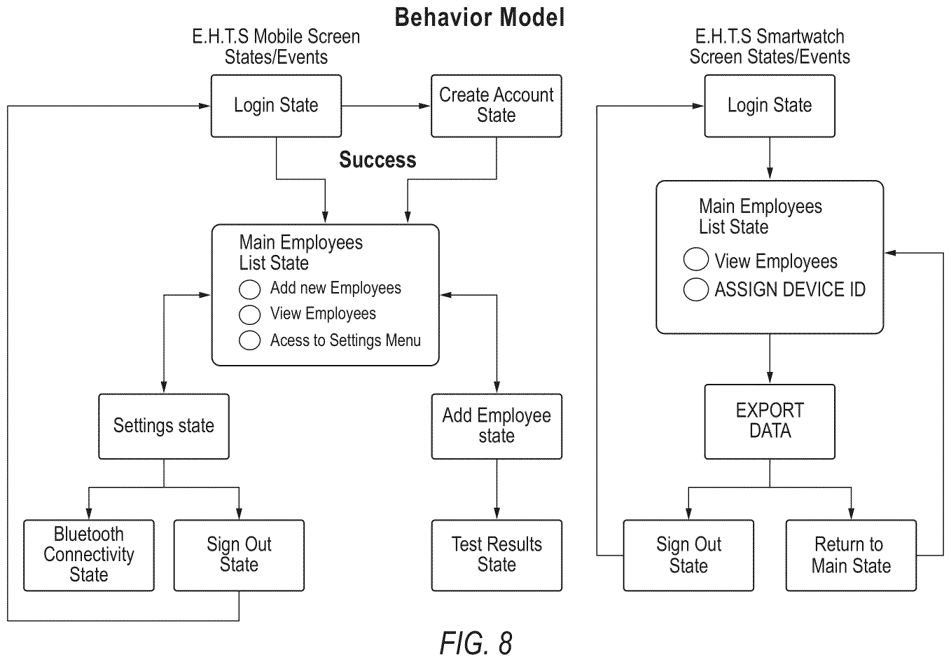

A behavior model of an embodiment of the EHTS mobile/cloud application is illustrated in FIG. 8. An example of events that may cause behavioral change in EHTS include creating a new account (new employee) or logging in via the login screen. In some embodiments, the user may input their email and password to begin using the EHTS application. After logging in successfully, the user (in some cases, a HR administrator) will be presented by EHTS with a main screen that allows the user to add a new employee through the profile screen, add additional employee personal information including name, age, employee ID number, height, weight, etc. Additionally, if the employee has used the EHTS previously, the user may access their test results using the test results screen. The EHTS application also allows for configurations such as Bluetooth® connectivity and a logout screen.

A behavior model of an embodiment of the EHTS smart watch application include logging in via the login screen on display 12 of smart watch 10. Employers or users are then required to enter their email and password (other login types may be used) that may have been created using the mobile/cloud EHTS application. In other embodiments, the employer or user may create a login using the smart watch EHTS application. Next, the display presents a main screen to allow the user to select an employee from the list of employees. EHTS then assigned a device ID to the user. The smart watch EHTS application allows for events such as exporting recorded data to a cloud-based system using the export data button on the smart watch, logging out of the EHTS application, or returning to the main screen. EHTS allows the user to configure settings on both the mobile/cloud application and the smart watch to add/delete employees, view employee data, retrieve recorded data, and testing new and existing employees.

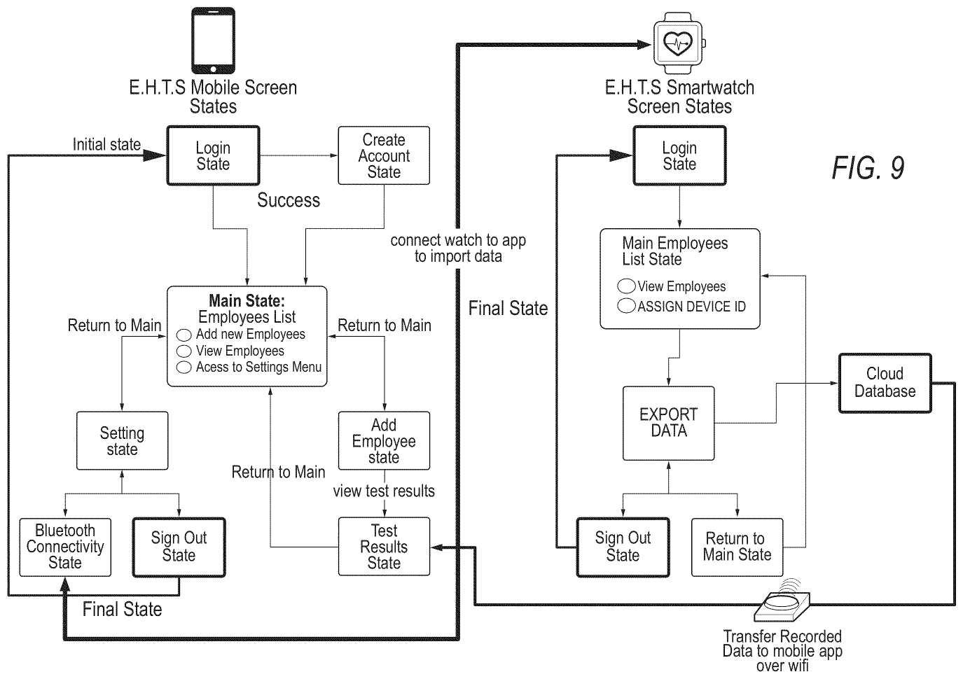

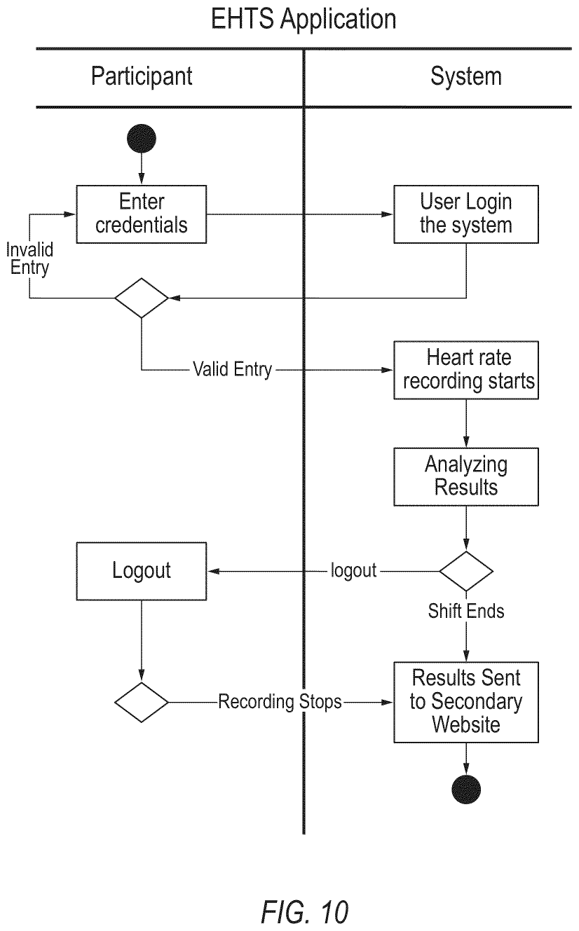

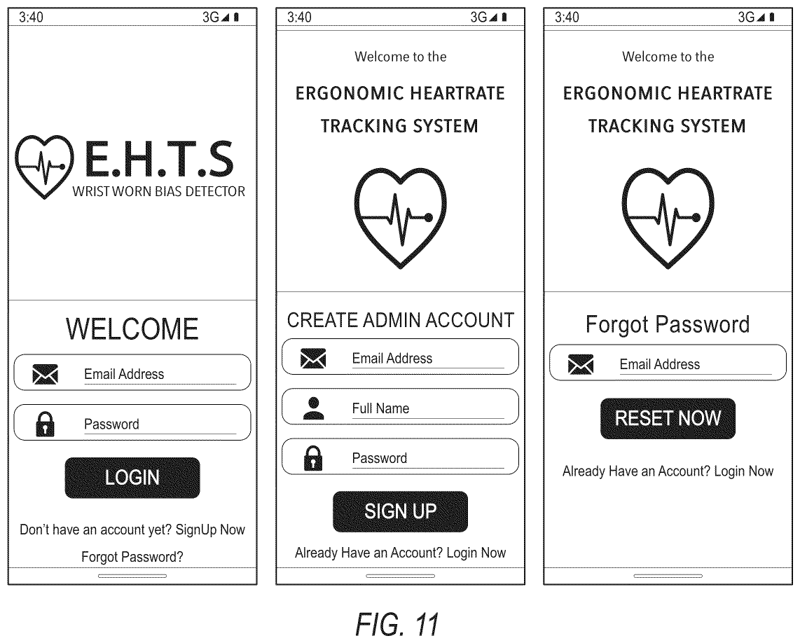

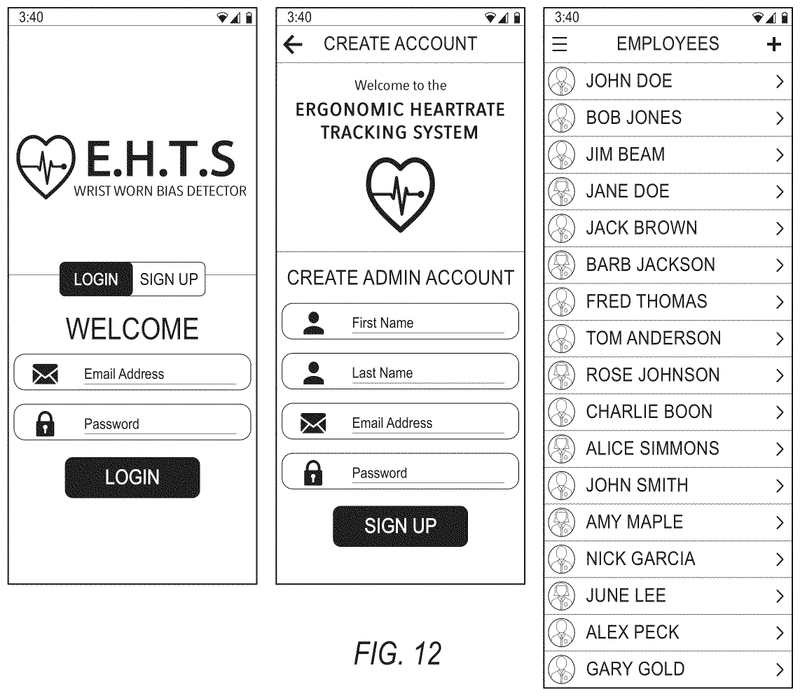

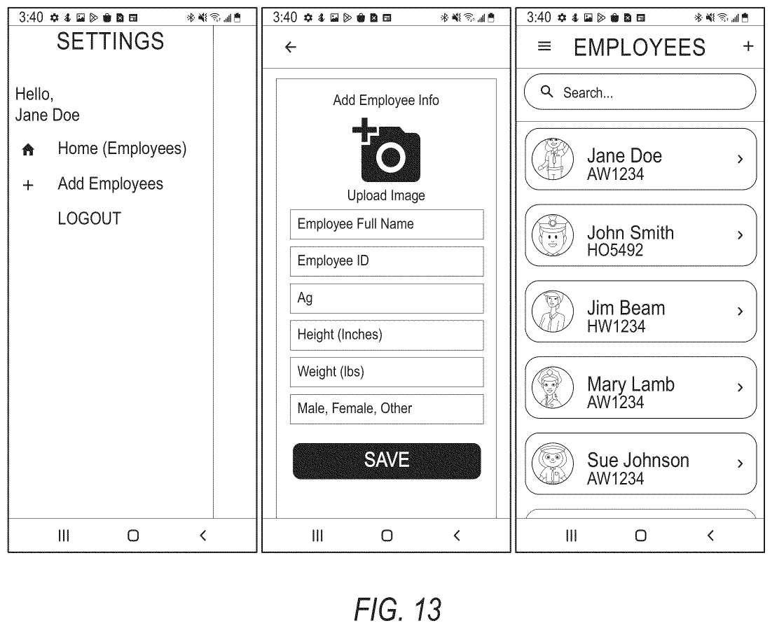

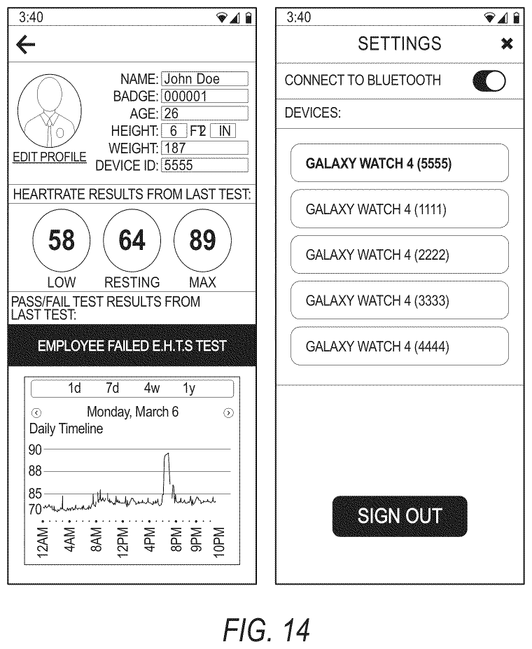

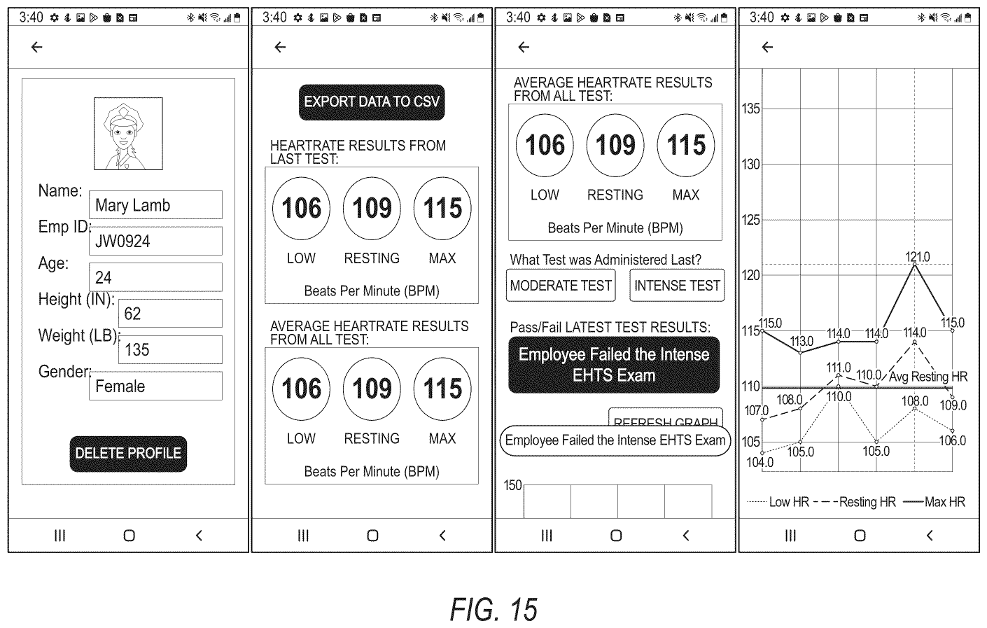

FIGS. 9-15 illustrate embodiments of behavior modes of the mobile/cloud EHTS application and the smart watch EHTS application.

For the mobile EHTS application, if the employer executes the EHTS mobile application, a user login screen is displayed allowing the user to enter their ID and password to access their user (admin) account. If the correct ID is entered, the EHTS displays the main screen including the employee list. If the incorrect ID is entered, a prompt for correct ID is displayed. If the user is new to the EHTS application, the user will be prompted by EHTS to create their new account. Once the user is logged into EHTS, the main screen including employees will be displayed, unless the EHTS application is new and no employees are currently stored in the EHTS. The main screen also includes additional buttons for customizing and adding employee data. To add a new employee profile, the user selects an add button to add the new employee to EHTS. The user may then add the appropriate information to the employee profile and save the information to the EHTS database. After the employee profile is saved, the user is then displayed test results, if any, for the employee. The test results will display the employee's resting heart rate, low and high heart rates, and any pass/fail test results, test information, and historical graphs. A Pass or Fail is shown where appropriate in the employee's profile. The user may select EHTS settings and connect to a Bluetooth® device such as EHTS smart watches 10. A sign out button is also displayed by the EHTS. Signing out will result in EHTS displaying the login screen.

For the smart watch EHTS application, the user (admin) opens the EHTS application on the smart watch 10, a login screen is shown on display 12 to allow the user to enter their login ID and password to access their account in EHTS. If the password and ID is entered correctly, the user is shown the main screen listing employees stored in EHTS. If the password and ID is not entered correctly, EHTS prompts the user to enter the correct information. After the user has correctly logged in the list of employees stored in EHTS is shown, if any. Navigation tools are displayed for the user to scroll through the employee list as well as enter new employee data. The user then selects a chosen employee to be biased tested by EHTS. A new data file is created to store the employee's heart rate and/or audio data for a test real-life situation or a simulated test for biases. When the test is completed, an export button will be displayed to the user by EHTS to transmit the employee's data to the EHTS mobile/cloud application. A logout button is then displayed to the user after the export of employee data or a return button, returning the user to the employee list to start a new bias test using the EHTS.

The preceding examples illustrate embodiments of the disclosure, but should not be viewed as limiting the scope of the disclosure.

Other embodiments and uses of the disclosure will be apparent to those skilled in the art from consideration of the specification and practice of the disclosure as disclosed herein. All references cited herein, including all publications, U.S. and foreign patents and patent applications, are specifically and entirely incorporated by reference. The term comprising, wherever used, is intended to include the terms consisting and consisting essentially of. Furthermore, the terms comprising, including, and containing are not intended to be limiting. It is intended that the specification and examples be considered exemplary only with the true scope and spirit of the disclosure indicated by the following claims.

1. A heart rate tracking and bias detecting smart watch system comprising:
a heart rate tracking and bias detecting smart watch housing configured to engage the wrist of a user, wherein the heart rate tracking smart watch housing includes a heart rate tracker, an audio recorder, a display, a wireless communicator, and a computing device including a processor and memory communicatively connected to the heart rate tracker, the audio recorder, the display, and the wireless communicator, wherein the computing device is configured to:
receive an input activating the device using the display;
record the heart rate data and the voice audio of the user in response to activation of the device;
transmit the recorded voice audio and the recorded heart rate data of the user to an external device application, wherein in response to the transmission, the external device application is configured to generate a report on the user, ascertain whether the user is suitable for a particular job based on predetermined bias criteria; and
receive an input deactivating the device using the display in response to the complete transmission of the recorded audio and the heart rate data to the application.

2. The heart rate tracking smart watch device of claim 1, wherein the computing device is further configured to execute a heart rate data calibration application when the device is activated using the display.

3. The heart rate tracking smart watch device of claim 2, wherein the computing device is further configured to display user information upon completion of the heart rate data calibration using the display.

4. The heart rate tracking smart watch device of claim 1, wherein the computing device is further configured to record the heart rate data of the user during a bias simulation and transmit the heart rate data to the application.

5. The heart rate heart rate tracking smart watch device of claim 1, wherein the computing device is further configured to analyze the user's heart rate data according to predetermined parameters including levels of heart rate activity and transmit the analyzed data to the application.

6. The heart rate heart rate tracking smart watch device of claim 5, wherein the computing device is further configured to analyze the user's heart rate data according to predetermined parameters including levels of heart rate activity and provide the analyzed data to the user using the display.

7. The heart rate tracking smart watch device of claim 1, wherein the computing device is further configured to provide the heart rate data to the user using the display.

8. The heart rate tracking smart watch device of claim 1, wherein the computing device is further configured to indicate to the user that audio is being recorded using the display.

9. The heart rate tracking smart watch device of claim 1, wherein the computing device is further configured to transmit the recorded audio and the recorded heart rate data of the user to a virtual reality simulation application.

10. The heart rate tracking smart watch device of claim 1, wherein the computing device is further configured to provide to the user confirmation of the complete transmission of the recorded audio and heart rate data to the application using the display.

11. The heart rate tracking smart watch device of claim 1, wherein the application further is configured to generate a folder for a new user to store to store the recorded audio and the recorded heart rate data of the user to the ergonomic heart rate tracking system.

12. The heart rate tracking smart watch device of claim 1, wherein the application further comprises external memory storage to record data of each user, wherein the data includes heart rate data and audio files of the voice of each user.

13. The heart rate tracking smart watch device of claim 1, wherein the external device application is configured to analyze the voice audio files of the user for variations in pitch.

14. The heart rate tracking smart watch device of claim 1, wherein the heart rate tracker includes a user heart rate calibration mode.

15. The heart rate tracking smart watch device of claim 1, wherein the computing device is further configured to execute situational simulations on the user.

16. The heart rate tracking smart watch device of claim 1, wherein the computing device is further configured to stealth mode, wherein the stealth mode includes a limited display output to the user.

17. The heart rate tracking smart watch device of claim 1, wherein the computing device is further configured with a location device to transmit the location of the heart rate smart watch device to the external device application.

18. The heart rate tracking smart watch device of claim 17, wherein the computing device is further configured to retrieve the status of the user from the external device application and selectively record the heart rate and voice audio of the user dependent upon that status.

19. A method comprising:
receiving, using a heart rate tracking and bias detecting smart watch device, an input from a user activating the device using the display, wherein the input includes at least one of a character or a user biometric authenticator;
recording, using the heart rate tracking smart watch device, the heart rate data and the audio of the user in response to activation of the device;
transmitting, using the heart rate tracking smart watch device, the recorded audio and the recorded heart rate data of the user to an application, wherein the application stores the data and generates a report on the user and ascertains whether the user is suitable for a particular job based on predetermined bias criteria; and
receiving, using the heart rate tracking smart watch device, an input from the user deactivating the device using the display in response to the complete transmission of the recorded audio and the heart rate data to the application.

20. A non-transitory computer-readable storage medium comprising instructions that cause one or more computing devices to:
receive, using a heart rate tracking and bias detecting smart watch device, an input from a user activating the device using the display, wherein the input includes at least one of a character or a user biometric authenticator;
record, using the heart rate tracking smart watch device, the heart rate data and the audio of the user in response to activation of the device;
transmit, using the heart rate tracking smart watch device, the recorded audio and the recorded heart rate data of the user to an application, wherein the application stores the data generates a report on the user and ascertains whether the user is suitable for a particular job based on predetermined bias criteria; and
receive, using the heart rate tracking smart watch device, an input from the user deactivating the device using the display in response to the complete transmission of the recorded audio and the heart rate data to the application.
